(19) United States
(12) Patent Application Publication
(10) Pub. No.: US 2023/0301878 A1
(43) Pub. Date: Sep. 28, 2023

(54) COATED POWDER FOR POWDER JET CLEANING OF TEETH

(71) Applicant: FERTON HOLDING S.A., DELÉMONT (CH)

(72) Inventors: Marcel DONNET, Saint Jean de Gonville (FR); Simone GATTI, Chavannes-près-Renens (CH); Oliver Martin ANTON, Biblis (DE)

(21) Appl. No.: 18/020,289

(22) PCT Filed: Aug. 10, 2021
(86) PCT No.: PCT/EP2021/072313

§ 371 Date: Feb. 8, 2023

(30) Foreign Application Priority Data
Aug. 11, 2020 (EP) 20190456.2

(51) Int. Cl. A61K 8/02; A61Q 11/00; A61K 8/34; A61K 8/25

(52) U.S. Cl. CPC A61K 8/0241; A61Q 11/00; A61K 8/345; A61K 8/25; A61K 2800/92; A61K 2800/412; A61K 2800/622; A61K 2800/28

(57) ABSTRACT

A powder for use in a powder jet device for cleaning tooth surfaces by powder spraying, where the powder comprises at least 90 wt.-% of particles of a first material and 0.1-10 wt.-% of a second material, where the second material is coated on a surface of the particles of the first material and a solubility of the first material in water is ≥1 g/L.

### TECHNICAL FIELD

The disclosure relates to a powder for use in a powder jet device for cleaning tooth surfaces by powder spraying. The disclosure further relates to the use of the powder in a powder jet device and to a process for cleaning teeth by using the powder.

### BACKGROUND

Professional dental prophylaxis is a maintenance treatment comprising powder jet cleaning or air polishing and aims to remove dental plaque and calculus that a patient is not able to remove with daily home care alone. Such powder jet cleaning is particularly effective as it allows to reach and clean all tooth surfaces and the inter-spaces between them as well as implants, brackets and appliances.

In the powder jet cleaning process, a powder is sprayed with a gaseous carrier medium, usually air, onto tooth surfaces, which allows an efficient cleaning of the teeth. Additionally or as an alternative to a gaseous carrier medium, a liquid carrier medium, for example water may be used. Powder jet cleaning is performed with a powder jet device and it is particularly effective because it does not require repetitive movements nor different stages. Further, it is faster than other cleaning methods and it needs relatively low training to be learned correctly. The cleaning occurs without damaging enamel dentine and soft tissues as long as the powder is sufficiently soft and the particles are sufficiently small.

DE 200 09 665 U1 discloses a dental cleaning powder containing a basic powder of sodium bicarbonate (sodium hydrogen carbonate), alternatively calcium carbonate or glycine and additional active ingredients such as anti-microbial compounds or ingredients which contribute to the remineralisation of the teeth.

EP 3 253 359 A1 discloses particles with an average particle size of around 10-100 μm for gentle and nevertheless efficient tooth cleaning. Small particles sizes are preferred for cleaning subgingival tooth surfaces and larger particle sizes are preferred for cleaning the supragingival tooth surfaces. Larger particle sizes usually result in discomfort or pain when subgingival tooth surfaces or soft tissues as gum are hit.

Important features of powders for use in dental cleaning are the hardness and the abrasiveness of the powders. A too hard and abrasive powder can damage the enamel and the composites used for dental reparation. Therefore, soft materials such as sodium bicarbonate are often used in powder jet devices. However, even with this material, patients often have discomfort and there is a risk of damaging soft tissues such as dentine and gingiva so that this technique was usually used only on enamel and in general supragingival. A further important feature of the cleaning powder is the cleaning efficiency. A lower abrasiveness of a powder is often accompanied by a lower cleaning efficiency. Therefore, a balance between abrasiveness and cleaning efficiency is desired and the optimum is a low abrasiveness and a still high or sufficient cleaning efficiency, if possible.

Efficient cleaning powders for use in powder jet devices are based on alditols, in particular mannitol and/or erythritol, as described in EP 2 228 175 A1.

A powder for subgingival treatment is based on glycine. Such glycine powders are less abrasive and thus safer and more comfortable for patients even when used on soft tissues such as gingiva and exposed dentine. However, a high decrease in cleaning efficiency is usually experienced, compared with the more abrasive sodium bicarbonate powders so that more time and a higher amount of powder is needed for the same cleaning result.

Another powder for tooth cleaning is based on erythritol, which is a material harder than glycine but softer than sodium bicarbonate. Erythritol powder is more efficient in powder jet cleaning than glycine and thus a lower time and lower amount of powder is required to perform a treatment. However, the abrasiveness is again slightly enhanced.

### BRIEF SUMMARY

In view of the above, there is still a need for dental cleaning powders with low abrasiveness and a still high cleaning efficiency.

Therefore, it is an object of the present disclosure to provide a powder for use in a powder jet device for cleaning tooth surfaces that overcomes the disadvantages of conventional powders, in particular a tooth cleaning powder with lower abrasiveness than known powders and a still good cleaning efficiency.

This object is achieved according to the disclosure by a powder for use in a powder jet device for cleaning tooth surfaces by powder spraying, wherein the powder comprises at least 90 wt.-%, based on the total weight of the powder, of particles of a first material and 0.1-10 wt.-%, based on the total weight of the powder, of a second material, wherein the second material is coated on the surface of the particles of the first material and wherein the solubility of the first material in water is 1 g/L (grams per liter).

The object is further achieved by a process for cleaning tooth surfaces, wherein this powder is sprayed with a powder jet device onto a tooth surface together with a gaseous and/or liquid carrier medium. The object is also achieved by the use of such powder or powder mixture in a powder jet device for cleaning tooth surfaces by powder spraying.

Preferred embodiments of the disclosure are subject to the dependent claims as well as the following description.

It has surprisingly been found that a relatively small amount of a second material that is coated on the particles of the first material provides a powder with a lower abrasiveness and simultaneously a good cleaning efficiency compared to a powder comprising the particles of the first material alone. Without being bound by this ex-planation, it is assumed that this result is achieved by buffering the impact of the particles with the coating. The coating breaks when the particles hit the tooth surface which absorbs energy resulting in a buffering effect. This reduces the abrasiveness, whereas the still high cleaning efficiency is a result of the particles of the first material that are still present after breaking of the coating. This effect is combined with a good solubility of 1 g/L of the first material in water, which gives an improved compatibility of the powder because inadvertently inhaled particles dissolve in the water film that is present in the lungs.

“Coated” or “coating” according to the disclosure means that the second material forms a layer or film on the surface of the particles of the first material. The second material may cover parts of the surface or the entire surface of the particles of the first material. The thickness of the coating may vary between the particles. The thickness is preferably 0.01-10 μm, more preferably 0.02-8 μm, and most preferably 0.03-5 μm.

A powder for use in a powder jet device for cleaning tooth surfaces by powder spraying according to the disclosure means a powder for use in cleaning tooth surfaces by powder spraying with a powder jet device.

Cleaning tooth surfaces within the meaning of the disclosure is the partial or complete removal of dental plaque and/or calculus from tooth surfaces. The cleaning is performed with conventional powder jet devices for cleaning teeth by powder spraying as described above.

The maximum particle size of the powder according to the disclosure (d₁₀₀) is preferably equal or less than 200 μm, more preferably 150 μm, even more preferably ≤120 μm and most preferably 100 μm, in order to achieve an efficient tooth cleaning. The average particle size (d₅₀) is preferably 5 to 75 μm, in particular 10 to 70 μm. The particle sizes may be adapted to the field of application of dental cleaning that is provided. For example, a smaller average particle size of the particles is preferred for cleaning subgingival tooth surfaces, in particular about 5 to 40 μm, more preferably 10 to 30 μm. For cleaning supragingival tooth surfaces, a larger average particle size is preferred, in particular about 20 to 75 μm, more preferably 30 to 70 μm.

In the context of this disclosure, the d₅₀ value is the particle size at which 50% of the particles are smaller than the d₅₀ value in terms of volume and 50% of the particles are larger than the d₅₀ value in terms of volume. This applies also to the d₁₀₀ values, the so-called maximum particle size. For a given d₁₀₀ value, 100% of the particles with regard to the volume are smaller than this value. A powder with a certain maximum particle size can be prepared by sieving the powder with a sieve having an appropriate mesh size. The d-values according to the disclosure are determined by laser diffraction using a dry dispersion unit (Malvern Mastersizer 2000, equipped with a Scirocco dry dispersion unit, operated at 1.5 bar).

The powder or powder mixture according to the disclosure comprises particles and these particles of the powder comprise particles of a first material coated with a second material. Accordingly, the particles of the first material are a little smaller than the particles of the powder because of the coating. The above particle sizes refer to the particles of the powder, i.e. the coated particles.

The powder according to the disclosure comprises at least 90 wt.-%, based on the total weight of the powder, of particles of the first material and 0.1-10 wt.-% of the second material. In a preferred embodiment of the disclosure, the powder comprises 90-99.9 wt.-% of a first material, based on the total weight of the powder, and 0.1-10 wt.-% of a second material, also based on the total weight of the powder. More preferably, the powder according to the disclosure comprises at least 93 wt.-%, preferably 93-99.8 wt.-% of a first material and 0.2-7 wt.-% of a second material.

Within the meaning of the present disclosure it should be understood that the amounts of the components of the powder, given in wt.-% (weight-%), sum up to 100%. By way of example, a powder comprising 95 wt.-% of a first material contains other components in a total amount of 5 wt.-%. The amount of 5 wt.-% can comprise, for example, 4 wt.-% of the second material and 1 wt.-% additional components.

As described, the surface of the first material can be partially or fully covered with the second material. Preferably, the second material covers at least 80% of the surface of the particles of the first material, more preferably at least 90% and most preferably at least 95% of the surface of the particles of the first material.

The first material has a good solubility in water of ≥1 g/L. The solubility refers to distilled water with a temperature of 20° C. and at a pressure of 1 bar. It is more preferred that the first material has a solubility in water of ≥2 g/L, even more preferred ≥3 g/L and most preferred ≥5 g/L. The advantage of a high or good solubility in water is that water-soluble materials cause less problems in the lungs if particles are inadvertently inhaled, because water-soluble substances dissolve in the water film that is present in the lungs.

Further, it is preferred that the second material has also a good solubility in water. In particular, it is preferred that the solubility of the second material in water is ≥1 g/L, more preferably ≥2 g/L, even more preferably ≥3 g/L and most preferably ≥5 g/L.

In a preferred embodiment of the disclosure, the first material is selected from the group consisting of glycine, sodium hydrogen carbonate, tagatose, trehalose, alditols and mixtures thereof. These materials are known as suitable materials for dental cleaning powders. More preferably, the first material is selected from the group consisting of glycine, alditols and mixtures thereof. Even more preferably, the first material is an alditol or a mixture of alditols.

The alditol according to the disclosure is preferably selected from the group consisting of erythritol, sorbitol, xylit (xylitol), mannit (mannitol), lactitol, threit (threitol), arabitol, and isomalt. Most preferably, the alditol is erythritol. Therefore, the first material is more preferably selected from the group consisting of glycine, sodium hydrogen carbonate, tagatose, trehalose, erythritol and mixtures thereof.

In a further preferred embodiment of the disclosure, the second material is also selected from the group consisting of glycine, sodium hydrogen carbonate, tagatose, trehalose, alditols and mixtures thereof, more preferably selected from the group consisting of glycine, sodium hydrogen carbonate, tagatose, trehalose, erythritol and mixtures thereof. The first material and the second material can be the same or different, which means that particles of a first material are coated with 0.1-10 wt.-% of the same material. Accordingly, the first material and the second material are selected from the group consisting of glycine, sodium hydrogen carbonate, tagatose, trehalose, alditols and mixtures thereof. The alditol is preferably erythritol.

It has been found that the first material and the second material are more preferably not made of the same material. The second material is even more preferably a protein, an amino acid, a vitamin, a polysaccharide, an organic acid, a peptide hormone, a natural compound, an alcohol, a quaternary amine, a biguanide, a sugar or mixtures thereof. The protein is preferably a collagen, in particular collagen type I or collagen type II, the amino acid is preferably asparagine or arginine, the vitamin is preferably vitamin C, the polysaccharide is preferably hyaluronic acid or cyclodextrin, the organic acid is preferably aspirin, the peptide hormone is preferably IGF-1 or IGF-2, the natural compound is preferably Aloe Vera extract or Propolis, the alcohol is preferably menthol, the quaternary amine is preferably cetylpyridinium chloride, the biguanide is preferably chlorhexidine, and the sugar is preferably rhamnose. More preferably, the second material is selected from the group consisting of asparagine, arginine, collagen, cyclodextrin and mixtures thereof. The collagen is preferably collagen type I or collagen type II. Preferred cyclodextrins are α-cyclodextrin, β-cyclodextrin and γ-cyclodextrin.

The second material is even more preferably selected from the group consisting of amino acid, alditol, sugar, cyclodextrin and mixtures thereof. The amino acid is preferably asparagine, arginine and/or alanine, more preferably asparagine and/or arginine. The alditol is preferably erythritol. The sugar is preferably rhamnose, tagatose and/or trehalose, more preferably rhamnose. The cyclodextrin can be α-, β-, γ- or δ-cyclodextrin. Preferred are α-, β- and/or γ-cyclodextrin. Even more preferably, the second material is selected from the group consisting of asparagine, arginine, alanine, erythritol, cyclodextrin, tagatose, trehalose, rhamnose and mixtures thereof. The second material is even more preferably selected from the group consisting of asparagine, arginine, alanine, erythritol, cyclodextrin and mixtures thereof. Asparagine, arginine, cyclodextrin and mixtures thereof are most preferred for the second material.

In a preferred embodiment of the disclosure, the first material is selected from the group consisting of glycine, sodium hydrogen carbonate, tagatose, trehalose, erythritol and mixtures thereof, and the second material is selected from the above described groups. In a further preferred embodiment, the first material is an alditol or a mixture of alditols, preferably erythritol, and the second material is selected from the above-described groups, in particular asparagine, arginine, cyclodextrin and mixtures thereof.

In a further preferred embodiment of the disclosure, the powder additionally comprises a flow aid, a bleaching agent, a bactericide, an analgesic and/or a flavouring agent. The total amount of these additional substances is preferably 0.2 to 5 wt.-%, based on the total weight of the powder, more preferably 0.5 to 2 wt.-%.

The flow aid is preferably selected from the group consisting of silicon dioxide (silica), aluminium silicate and/or aluminium hydroxide. Silicon dioxide is more preferred, in particular in an amount of 0.2 to 3 wt.-%, most preferably 0.5 to 2 wt.-%, based on the total weight of the powder.

Preferred bleaching agents are peroxides such as magnesium, calcium or zinc peroxides, persulfates such as sodium, potassium or ammonium persulfates or perborates.

Preferred bactericides are chlorhexidine, chlorhexidine digluconate, triclosan, cetylpyridinium chloride, hexetidine, tin(II) salts and zinc salts. Preferred analgesics are lidocaine and articaine.

It is also preferred that the powder for use in a powder jet device according to the disclosure consists of the described components. By way of example, a preferred powder consists of 90-99.9 wt.-% of a first material, preferably selected from the group consisting of glycine, sodium hydrogen carbonate, tagatose, trehalose, alditols and mixtures thereof, and 0.1-10 wt.-% of a second material, preferably selected from the group consisting of asparagine, arginine, collagen, cyclodextrin and mixtures thereof, and optionally 0.2-3 wt.-% of a flow aid, in particular silicon dioxide, further optionally 0.2 to 5 wt.-% of a bactericide, a bleaching agent and/or an analgesic.

The disclosure also relates to the use of the powder or powder mixture according to the disclosure in a powder jet device for cleaning tooth surfaces by powder spraying. The Powder or powder mixture as used in a powder jet device comprises at least 90 wt.-%, based on the total weight of the powder, of particles of a first material and 0.1-10 wt.-%, based on the total weight of the powder, of a second material, wherein the second material is coated on the surface of the particles of the first material, wherein the first and second materials are different, and wherein the first material is selected from the group consisting of glycine, sodium hydrogen carbonate, tagatose, trehalose, alditols, and mixtures thereof.

The preferred embodiments of the powder described herein are also preferred for the use of the powder or powder mixture.

The disclosure further relates to a process for cleaning tooth surfaces wherein the powder according to the disclosure is sprayed with a powder jet device onto the tooth surfaces together with a gaseous carrier medium and/or a liquid carrier medium, in particular air and optionally a fluid such as water.

### DESCRIPTION AND EXAMPLES

The following examples provide preferred embodiments according to the disclosure.

### EXAMPLES

### Example 1: Erythritol Coated with 1 wt.-% Asparagine

The experiment was carried out in a pilot batch fluidized bed of conical shape. Initial particle mass 1 kg, inlet air temperature 60-90° C., liquid feed rate 10 ml min⁻¹ and relative air spraying pressure 2-3.5 bar were chosen as process parameters. A solution of 2.5% L-asparagine was sprayed for 40 min onto the erythritol particles. During the experiment, all process parameters (air inlet temperature, air flow rate, liquid feed rate and spraying pressure) were kept constant. At the end of the experiment the coated powder was removed and the solid content checked to be more than 98%. The amount of asparagine added to the powder was about 1%. The resulting mean particle size was 20 microns.

### Example 2: Erythritol Coated with 0.5 wt.-% Collagen Type II

The experiment was carried out in a pilot batch fluidized bed of conical shape. Initial particle mass 1 kg, inlet air temperature 60-90° C., liquid feed rate 10 ml min⁻¹ and relative air spraying pressure 1-2.5 bar were chosen as process parameters. A solution of 2% Collagen was sprayed for 20 min. During the experiment, all process parameters (air inlet temperature, air flow rate, liquid feed rate and spraying pressure) were kept constant. At the end of the experiment the coated powder was removed and the solid content checked to be more than 98%. The amount of collagen added to the powder was about 0.5%. The resulting mean particle size was 15 micron.

The two powders according to Example 1 and Example 2 were tested regarding cleaning efficiency and abrasiveness, together with an erythritol powder coated with 10 wt.-% erythritol. The results are shown in FIGS. 1 and 2 in comparison to erythritol powder.

The efficiency or cleaning efficiency is the surface that is cleaned per gram of the powder.

The abrasiveness is determined by projecting powder with a powder jet device directly on a surface at 45° and 2 mm of projected distance using an EMS Airflow® prophylaxis master device. This surface is made of pure aluminium (99.5%). The application time is 30 seconds. The plate is put on an elevator to reach the distance of 2 mm. The nozzle is fixed in a resin mould in order to fix well the nozzle position. The mass is known by weighing the powder chamber before and after the test. Every measurement is repeated at least three times and the average is taken as abrasiveness. The depth of the holes was measured by a laser profilometer.

The average particle size of the powders according to the disclosure was determined in a Malvern Mastersizer 2000 (Malvern Instruments Ltd., Malvern, United Kingdom) with a scirocco dispersing unit at a dispersion pressure of 1.5 bar.

### Example 3: Erythritol and Glycine with Different Coatings

In accordance to the above experiments, erythritol and glycine were coated with different coatings as shown in FIGS. 3 and 4. The abrasiveness was determined as above described.

### BRIEF DESCRIPTION OF THE DRAWINGS

The disclosure is described further below with reference to FIGS. 1 to 4.

FIG. 1 shows the cleaning efficiency [mm²/g] of erythritol, compared to erythritol coated with 10% erythritol, erythritol coated with 1% asparagine and erythritol coated with 0.5% collagen.

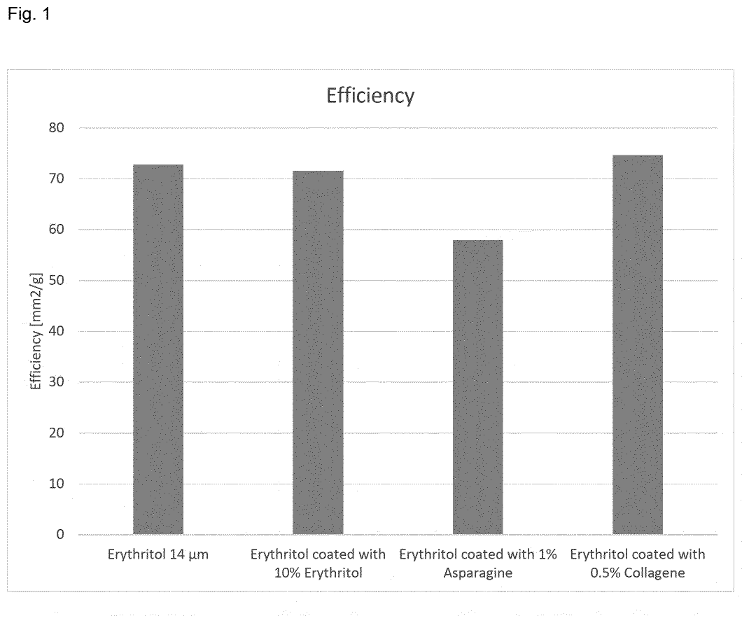

FIG. 2 shows the abrasiveness [μm/g] of erythritol powder compared to erythritol powder coated with 10% erythritol, erythritol coated with 1% asparagine and erythritol coated with 0.5% collagen.

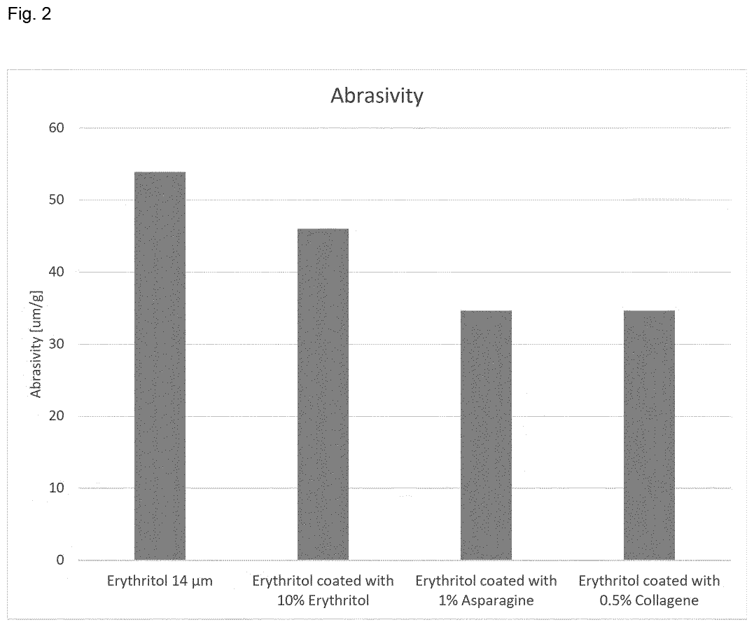

FIG. 3 shows the abrasiveness of erythritol powder coated with different coatings of arginine, cyclodextrin, arginine+cyclodextrin, asparagine, collagen, vitamin C, erythritol, alanine and rhodamine relative to erythritol powder without coating. It can be seen that erythritol coated with erythritol, arginine, alanine, cyclodextrin, arginine+cyclodextrin and asparagine have a reduced abrasiveness compared to erythritol as such and compared to erythritol+collagen, erythritol+vitamin C and erythritol+rhodamine. This reduced abrasiveness is advantageous, for example over erythritol coated with collagen, erythritol coated with vitamin C and erythritol coated with rhodamine.

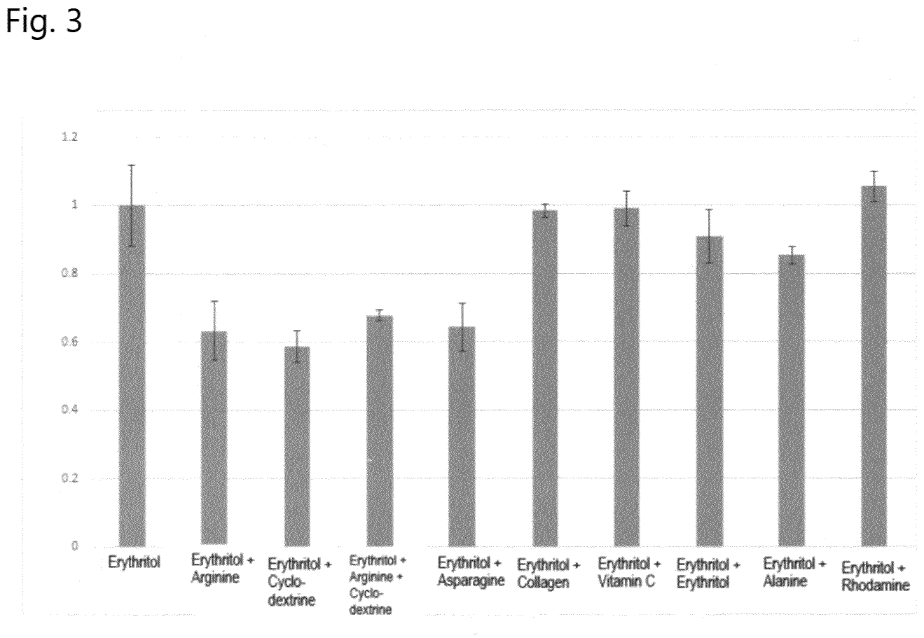

FIG. 4 shows the abrasiveness of glycine coated with cyclodextrin relative to glycine powder without coating.

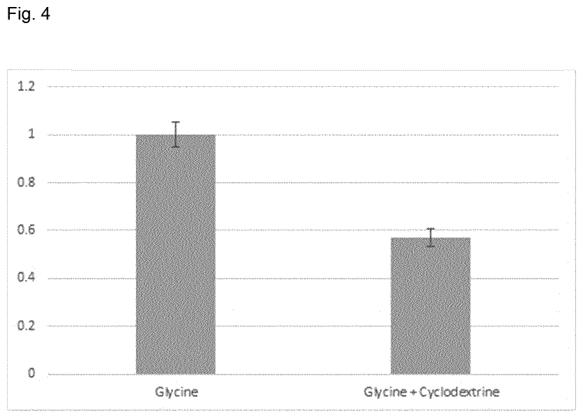

It has been demonstrated that the powders according to disclosure, wherein a second material is coated on particles of a first material show a reduced abrasiveness compared to powders without coating and still have a good cleaning efficiency. The ratio of efficiency vs. abrasivity makes the coated powders according to the disclosure more powerful than standard erythritol or glycine powders.

1. Powder for use in a powder jet device for cleaning tooth surfaces by powder spraying, the powder comprising:
at least 90 wt.-%, based on a total weight of the powder, of particles of a first material; and
0.1-10 wt.-%, based on the total weight of the powder, of a second material,
wherein the second material is coated on a surface of the particles of the first material and a solubility of the first material in water is ≥1 g/L.

2. Powder according to claim 1, wherein the powder comprises at least 93 wt.-% of the first material and 0.2-7 wt.-% of the second material, based on the total weight of the powder.

3. Powder according to claim 1, wherein the second material covers at least 80% of the surface of the particles of the first material.

4. Powder according to claim 1, wherein the solubility of the second material in water is ≥1 g/L.

5. Powder according to claim 1, wherein the particles of the powder have an average particle size (d₅₀) of 5-75 μm.

6. Powder according to claim 1, wherein the first material is selected from the group consisting of glycine, sodium hydrogen carbonate, tagatose, trehalose, alditols, cyclodextrin and mixtures thereof.

7. Powder according to claim 6, wherein the first material is an alditol.

8. Powder according to claim 1, wherein the second material is selected from the group consisting of glycine, sodium hydrogen carbonate, tagatose, trehalose, cyclodextrin, alditols and mixtures thereof.

9. Powder according to claim 1, wherein the second material is selected from the group consisting of amino acid, alditol, sugar, cyclodextrin and mixtures thereof.

10. Powder according to claim 9, wherein the second material is selected from the group consisting of asparagine, arginine, alanine, erythritol, tagatose, trehalose, rhamnose, cyclodextrin and mixtures thereof.

11. Powder according to claim 1, wherein the second material is selected from the group consisting of asparagine, arginine, cyclodextrin and mixtures thereof.

12. Powder according to claim 1, wherein maximum particle size (d₁₀₀) of the powder is ≤150 μm.

13. Powder according to claim 1, wherein the powder further comprises 0.5 to 2 wt.-% amorphous silicon dioxide.

14. Process for cleaning tooth surfaces, wherein a powder according to claim 1 is sprayed with a powder jet device onto a tooth surface together with a gaseous carrier medium and/or liquid carrier medium.

15. Powder according to claim 1, configured to be used in a powder jet device for cleaning tooth surfaces by powder spraying.
